(19) United States
(12) Patent Application Publication
(10) Pub. No.: US 2022/0340589 A1
(43) Pub. Date: Oct. 27, 2022

(54) METHODS OF SYNTHESIZING HIGH-PURITY CANNABICYCLOL AND ARTIFICIAL RESINS COMPRISING CANNABICYCLOL

(71) Applicant: Canopy Growth Corporation, Smiths Falls (CA)

(72) Inventors: Daniel BRUMAR, Smiths Falls (CA); Ben GEILING, Smiths Falls (CA); Mohammadmehdi HAGHDOOST MANJILI, Smiths Falls (CA); Mark ZOLKIEWSKI, Smiths Falls (CA)

(21) Appl. No.: 17/639,283

(22) PCT Filed: Dec. 24, 2020
(86) PCT No.: PCT/CA2020/051801

§ 371 Date: Feb. 28, 2022

Related U.S. Application Data
(60) Provisional application No. 62/987,179, filed on Mar. 9, 2020.
(60) Provisional application No. 62/953,370, filed on Dec. 24, 2019.

(51) Int. Cl. C07D 493/18

(52) U.S. Cl. CPC C07D 493/18

(57) ABSTRACT

Compositions having enhanced cannabicyclol (CBL) or CBL derivative concentrations are disclosed herein as are methods of synthesizing CBL and CBL derivative in high-purity form. Relative to conventional methods, the methods of the present disclosure may: (i) be better suited to large-scale conditions in that they do not require dangerous and/or toxic solvents and/or reagents; (ii) be more tolerant of complex starting compositions, such as cannabinoid extracts, isolates and/or distillates; (iii) provide CBL and/or CBL derivative at higher yield; (iv) provide easier methods to purify product mixtures comprising CBL and/or CBL derivative; (v) provide product mixtures that comprise unique ratios of CBL or CBL derivative relative to other cannabinoids; (vi) provide product mixtures with reduced THC concentrations and/or (vii) provide artificial resins having of a mixture cannabinoids that cannot be produced by extracting cannabis plant material.

### CROSS-REFERENCE TO RELATED APPLICATIONS

This application claims priority to and benefit of U.S. Provisional Patent Application Ser. No. 62/953,370 filed on Dec. 24, 2019 and U.S. Provisional Patent Application Ser. No. 62/987,179 filed on Mar. 9, 2020, each of which is hereby incorporated by reference in its entirety.

### TECHNICAL FIELD

The present disclosure generally relates to methods of synthesizing cannabinoids in high-purity forms and to methods for synthesizing cannabinoid compositions having unique ratios of cannabinoids. In particular, the present disclosure relates to methods for synthesizing high-purity cannabicyclol (CBL) and CBL derivatives and to cannabinoid compositions having enhanced CBL and/or CBL derivative concentrations.

### BACKGROUND

Cannabicyclol (CBL) is a cannabinoid found in the cannabis sativa plant, and it is quickly gaining consumer and scientific interest. For example, scientific studies indicate that CBL has the potential for anti-inflammatory and anti-tumour activity. CBL is also known to provide a variety of benefits in association with other cannabinoids via the entourage effect. CBL also has utility as a synthon for chemical applications. In spite of this wide-ranging potential, CBL is not currently utilized at scale. CBL is typically found in low concentrations in cannabis plant material, extracts, distillates and/or the like. At the same time, separating CBL from such compositions can pose significant challenges as CBL has similar physical characteristics (e.g. solubility and/or affinity profile) to a number of other cannabinoids. Furthermore, mixtures of cannabinoids having CBL at a concentration (absolute or relative to other cannabinoids) not found in cannabis plant material, extracts, distillates, and/or the like may be desirable.

For similar reasons, CBL can be difficult to separate from reaction mixtures. Conventional synthetic methods for producing CBL typically lack selectivity and lead to significant quantities of other products, such as tetrahydrocannabinol (THC). Isolating CBL from reaction mixtures can pose significant challenges, as CBL is difficult to separate from other products by typical purification methods (e.g. chromatography). This can make CBL difficult to obtain in pure form. THC is psychoactive, and compositions containing THC may be subject to regulatory restrictions or prohibitions on transportation or sale. This has the potential to limit the utility of the CBL compositions, because of the difficult associated with separating the two cannabinoids. This limitation is often compounded by the lack of scalability of conventional methods for synthesizing CBL.

For at least the foregoing reasons, methods for producing CBL and CBL derivatives in high-purity forms are desirable as are cannabinoid compositions with enhanced CBL and/or CBL derivative concentrations.

### SUMMARY

The present disclosure is based on extensive research and development directed at overcoming at least some of the current impediments to advancing the start of the art in CBL-related applications. As exemplified by the examples set out herein, the present disclosure advances this field with the provision of methods of synthesizing CBL and derivatives thereof in high-purity form. The present disclosure also provides cannabinoid compositions having unique ratios of CBL or CBL derivative to other cannabinoids. The methods of the present disclosure may be better suited to industrial scale in that they do not require dangerous and/or toxic solvents and/or reagents. The methods of the present disclosure may provide for more efficient purification of CBL and CBL derivatives. The methods of the present disclosure may provide access to: (i) CBL and CBL derivatives in high purity; (ii) mixtures of cannabinoids, including mixtures of CBL and cannabichromene (CBC), and derivatives thereof, that have unique cannabinoid ratios; (iii) mixtures of cannabinoids with reduced THC concentrations and/or (iii) artificial resins comprising mixtures of cannabinoids in proportions that cannot be produced by extracting cannabis plant material. Importantly, the present disclosure also provides synthetic methods that may be tolerant to a variety of complex starting compositions, such as cannabis extracts, isolates, and/or distillates.

Select methods of the present disclosure allow for conversion of CBC to CBL, or CBC derivatives to CBL derivatives. Without being bound to any particular theory, the present disclosure asserts that the ability to convert CBC into CBL as demonstrated herein is associated with intramolecular cyclization. In particular, the examples of the present disclosure indicate that the intramolecular cyclization can be induced by contacting the CBC with an acidic heterogeneous catalyst, contacting the CBC with a radical initiator, and/or by irradiating the CBC with UV light. The methods of the present disclosure may provide access cannabinoid compositions with unique ratios of CBL to CBC (or derivatives thereof). Select methods of the present disclosure allow for synthesizing CBL or a CBL derivative from citral and a modified resorcinol.

Select methods of the present disclosure allow for producing artificial resins from a starting material having a known composition. The starting material may include, for example, synthetic CBC and/or CBC isolate. Without being bound to any particular theory, the present disclosure asserts that conversion of at least a portion of the starting material to CBL provides access to the artificial resin that includes a mixture of cannabinoids with concentrations (relative or absolute) that cannot be produced by the extraction of cannabis plant material.

In select embodiments, the present disclosure relates to a method for converting CBC or a CBC derivative to CBL or a CBL derivative, the method comprising contacting the CBC or CBC derivative with an acidic heterogeneous material to form a product mixture comprising the CBL or CBL derivative.

In select embodiments, the present disclosure relates to a method for converting CBC or a CBC derivative to CBL or a CBL derivative, the method comprising contacting the CBC or CBC derivative with a radical initiator to form a product mixture comprising the CBL or CBL derivative.

In select embodiments, the present disclosure relates to a method for converting CBC or a CBC derivative to CBL or a CBL derivative, the method comprising irradiating the CBC or CBC derivative with UV light to form a product mixture comprising CBL or CBL derivative.

In select embodiments, the present disclosure relates to a method of preparing CBL or a CBL derivative, the method comprising: heating a reaction mixture comprising citral, a modified resorcinol, and an amine to form a first product mixture; and converting the first product mixture into a second product mixture by: (i) contacting the first product mixture with an acidic heterogeneous material to form the CBL or CBL derivative; (ii) contacting the first product mixture with a radical initiator to form the CBL or CBL derivative; or (iii) irradiating the first product mixture with UV light to form the CBL or CBL derivative.

In select embodiments, the present disclosure relates to a cannabinoid composition comprising (i) at least about 4% w/w CBL or a CBL derivative and (ii) at least about 4% w/w CBC or a CBC derivative.

In select embodiments, the present disclosure relates to a method of producing an artificial resin, the method comprising: contacting a starting material comprising CBC or a CBC derivative with an acidic heterogeneous material to form an artificial resin comprising at least about 4% w/w CBL or CBL derivative, a first cannabinoid, and a second cannabinoid.

In select embodiments, the present disclosure relates to a method of producing an artificial resin, the method comprising: contacting a starting material comprising CBC or a CBC derivative with a radical initiator to form an artificial resin comprising at least about 4% w/w CBL or CBL derivative, a first cannabinoid, and a second cannabinoid.

In select embodiments, the present disclosure relates to a method of producing an artificial resin comprising CBL or a CBL derivative, the method comprising: heating a reaction mixture comprising citral, a modified resorcinol, and an amine to form a product mixture; and converting the product mixture into the artificial resin by: (i) contacting the product mixture with an acidic heterogeneous material to form the CBL or CBL derivative; (ii) contacting the product mixture with a radical initiator to form the CBL or CBL derivatice; or (iii) irradiating the product mixture with UV light to form the CBL or CBL derivative.

In select embodiments, the present disclosure relates to an artificial resin comprising at least about 4% CBL or a CBL derivative, a first cannabinoid, and a second cannabinoid

Other aspects and features of the present disclosure will become apparent to those ordinarily skilled in the art upon review of the following description of specific embodiments.

### BRIEF DESCRIPTION OF THE DRAWINGS

FIG. 1 shows a high-performance liquid chromatogram for EXAMPLE 1;

FIG. 2 shows a high-performance liquid chromatogram for EXAMPLE 3;

FIG. 3 shows a high-performance liquid chromatogram for EXAMPLE 4;

FIG. 4 shows a high-performance liquid chromatogram for EXAMPLE 6;

FIG. 5 shows a high-performance liquid chromatogram for EXAMPLE 7;

FIG. 6A shows a perspective view of a crystal structure for CBL;

FIG. 6B shows a perspective view ORTEP drawing of a crystal structure for CBL;

FIG. 6C shows a stereoscopic view ORTEP drawing of a crystal structure for CBL;

FIG. 7 shows high-performance liquid chromatograms for EXAMPLE 9.

### DETAILED DESCRIPTION

As noted above, the present disclosure provides methods of synthesizing cannabicyclol (CBL) and derivatives thereof in high-purity forms. Relative to conventional methods, the methods of the present disclosure may: (i) be better suited to large-scale conditions in that they do not require dangerous and/or toxic solvents and/or reagents; (ii) be more tolerant of complex starting compositions, such as cannabinoid isolates and/or distillates; (iii) provide CBL and CBL derivative at higher yield; (iv) provide easier methods to purify product mixtures comprising CBL or CBL derivatives; (v) provide product mixtures that comprise unique ratios of CBL or CBL derivatives relative to other cannabinoids; (vi) provide product mixtures with reduced THC concentrations and/or; (vii) provide artificial resins having of a mixture cannabinoids that cannot be produced by extracting cannabis plant material. The present disclosure asserts that access to CBL and derivatives thereof via methods disclosed herein may be desirable in both medicinal and recreational contexts. Moreover, the present disclosure asserts that access to CBL and derivatives thereof via the methods disclosure herein is desirable to synthetic chemists.

The present disclosure provides cannabinoid compositions having unique ratios of cannabinoids, including unique ratios of cannabichromene (CBC) to CBL. Mixtures of cannabinoids may provide enhanced medicinal and/or recreational effects, for example via the entourage effect. The present disclosure provides cannabinoid compositions having reduced THC concentrations which may thereby avoid being subject to regulatory restrictions or prohibitions on transportation or sale. In some embodiments, the methods of the present disclosure provide access to high purity CBL and derivatives thereof, for example by converting CBC or a CBC derivative to CBL or a CBL derivative. High purity CBL or CBL derivative may be employed as an active pharmaceutical ingredient (API) for recreational and/or medicinal formulations, or as a synthon for chemical applications. High purity CBL or CBL derivative may be essentially free of THC, and thereby avoid being subject to regulatory restrictions or prohibitions on transportation or sale. In some embodiments, the methods of the present disclosure may employ a cannabis extract or a synthesis reaction mixture as a starting material, as the methods of the present disclosure may be compatible with impure starting materials.

Without being bound to any particular theory, the present disclosure asserts that converting CBC into CBL as demonstrated herein results from an intramolecular cyclization reaction. The intramolecular cyclization of CBC may be performed with the addition of a catalyst, reagent, UV light, or a combination thereof. In particular, the examples of the present disclosure indicate that the intramolecular cyclization can be induced by contacting the CBC with an acidic heterogeneous catalyst, a radical initiator, and/or by irradiating the CBC with UV light.

In the context of the present disclosure, the term “artificial resin” refers to a mixture of cannabinoids with a defined composition produced from starting materials comprising one or more of the same or different cannabinoids. In an embodiment, the starting material has a known composition (e.g. known cannabinoid content). Artificial resins may include 2, 3, 4, or more cannabinoids. Without being bound to any particular theory, the present disclosure asserts that using starting materials with known composition provides increases the reproducibility of the artificial resins produced. In contrast, producing a mixture of cannabinoids from cannabis plant extracts may reduce the reproducibility of the resulting mixture of cannabinoids due to the batch-to-batch variation of cannabis plant extracts. Non-exclusive examples of starting materials with a known composition include synthetic CBC or CBC derivative, CBC isolate, CBC derivative isolate, and mixtures of citral and a modified resorcinol (e.g. olivetol). The CBC isolate may be derived from cannabis plant material so long as the CBC isolate is sufficiently pure to ensure batch-to-batch consistency.

Without being bound to any particular theory, the present disclosure asserts that conversion of at least a portion of the starting material to CBL or CBL derivative provides access to artificial resins that include a mixture of cannabinoids with concentrations (relative or absolute) that cannot be produced by the extraction of cannabis plant material. For example, an artificial resin including CBL at concentrations (relative to other cannabinoids) higher than the concentrations of CBL found in cannabis plant material.

As used herein, the term “cannabinoid” refers to: (i) a chemical compound belonging to a class of secondary compounds commonly found in plants of genus cannabis, (ii) synthetic cannabinoids and any enantiomers thereof; and/or (iii) one of a class of diverse chemical compounds that may act on cannabinoid receptors such as CBI and CB2 . . . .

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In select embodiments of the present disclosure, the cannabinoid is a compound found in a mammal, sometimes called an endocannabinoid.

In select embodiments of the present disclosure, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5 diarylpyrazoles, quinolines, and arylsulfonamides as well as eicosanoids related to endocannabinoids.

In many cases, a cannabinoid can be identified because its chemical name will include the text string “*cannabi*”. However, there are a number of cannabinoids that do not use this nomenclature, such as for example those described herein.

In the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids.

As well, any and all isomeric, enantiomeric, or optically active derivatives are also encompassed. In particular, where appropriate, reference to a particular cannabinoid incudes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Examples of cannabinoids include, but are not limited to: cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic Acid (CBCVA), cannabichromevarin (CBCV), cannabidiolic acid (CBDA), cannabidiol (CBD), 06-cannabidiol (A6-CBD), cannabidiol monomethylether (CBDM), cannabidiol-C4 (CBD-C4), cannabidivarinic Acid (CBDVA), cannabidivarin (CBDV), cannabidiorcol (CBD-C1), tetrahydrocannabinolic acid A (THCA-A), tetrahydrocannabinolic acid B (THCA-B), tetrahydrocannabinol (THC or Δ9-THC), Δ8-tetrahydrocannabinol (Δ8-THC), trans-Δ10-tetrahydrocannabinol (trans-Δ10-THC), cis-A10-tetrahydrocannabinol (cis-Δ10-THC), tetrahydrocannabinolic acid C4 (THCA-C4), tetrahydrocannbinol C4 (THC-C4), tetrahydrocannabivarinic acid (THCVA), tetrahydrocannabivarin (THCV), Δ8-tetrahydrocannabivarin (Δ8-THCV), Δ9-tetrahydrocannabivarin (Δ9-THCV), tetrahydrocannabiorcolic acid (THCA-C1), tetrahydrocannabiorcol (THC-C1), 07-cis-iso- tetrahydrocannabivarin, Δ8-tetrahydrocannabinolic acid (Δ8-THCA), Δ9-tetrahydrocannabinolic acid (Δ9-THCA), cannabicyclolic acid (CBLA), cannabicyclol (CBL), cannabicyclovarin (CBLV), cannabielsoic acid A (CBEA-A), cannabielsoic acid B (CBEA-B), cnnabielsoin (CBE), cannabinolic acid (CBNA), cannabinol (CBN), cannabinol methylether (CBNM), cannabinol-C4 (CBN-C4), cannabivarin (CBV), cannabino-C2 (CBN-C2), cannabiorcol (CBN-C1), cannabinodiol (CBND), cannabinodivarin (CBDV), cannabitriol (CBT), 11-hydroxy-Δ9-tetrahydrocannabinol (11-OH-THC), 11-nor-9-carboxy- Δ9-tetrahydrocannabinol, ethoxy-cannabitriolvarin (CBTVE), 10 ethoxy-9-hydroxy-Δ6a-tetrahydrocannabinol, cannabitriolvarin (CBTV), 8,9 dihydroxy-A6a(10a)-tetrahydrocannabinol (8,9-Di-OH-CBT-05), dehydrocannabifuran (DCBF), cannbifuran (CBF), cannabichromanon (CBCN), cannabicitran, 10-oxo-A6a(10a)-tetrahydrocannabinol (OTHC), Δ9-cis-tetrahydrocannabinol (cis-THC), cannabiripsol (CBR), 3,4,5,6-tetrahydro-7-hydroxy-alpha-alpha-2-trimethyl-9-n-propyl-2,6-methano-2h-1-benzoxocin-5-methanol (OH-iso-HHCV), trihydroxy-delta-9-tetrahydrocannabinol (triOH-THC), yangonin, epigallocatechin gallate, dodeca-2e, 4e, 8z, 10z-tetraenoic acid isobutylamide, hexahydrocannibinol, and dodeca-2e,4e-dienoic acid isobutylamide.

In some embodiments of the present disclosure, the cannabinoid is a cannabinoid dimer. The cannabinoid may be a dimer of the same cannabinoid (e.g. THC-THC) or different cannabinoids. In an embodiment of the present disclosure, the cannabinoid may be a dimer of THC, including for example cannabisol.

As used herein, the term “THC” refers to tetrahydrocannabinol. “THC” is used interchangeably herein with “Δ9-THC”.

In an embodiment of the present disclosure, the cannabinoid is THC (Δ9-THC), Δ8-THC, trans-Δ10-THC, cis-Δ10-THC, THCA, THCV, Δ8-THCA, Δ9-THCA, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, CBLVA, CBT, cannabicitran, or any combination thereof.

Structural formulae of cannabinoids of the present disclosure may include the following:

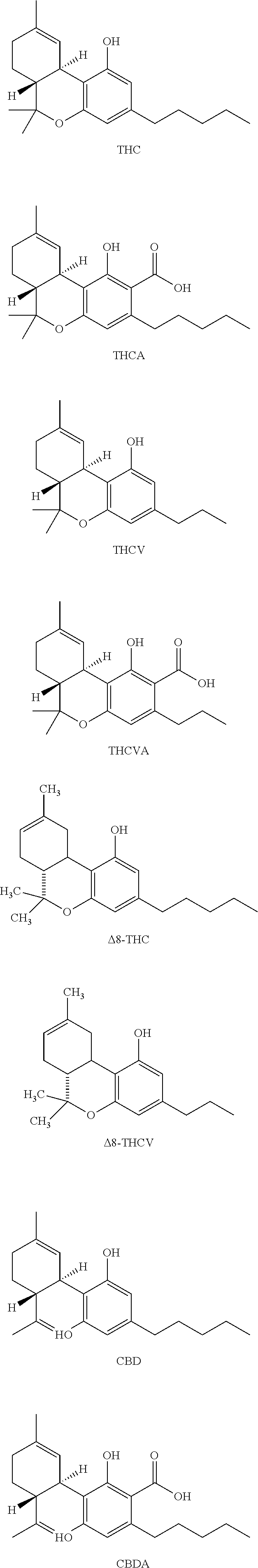

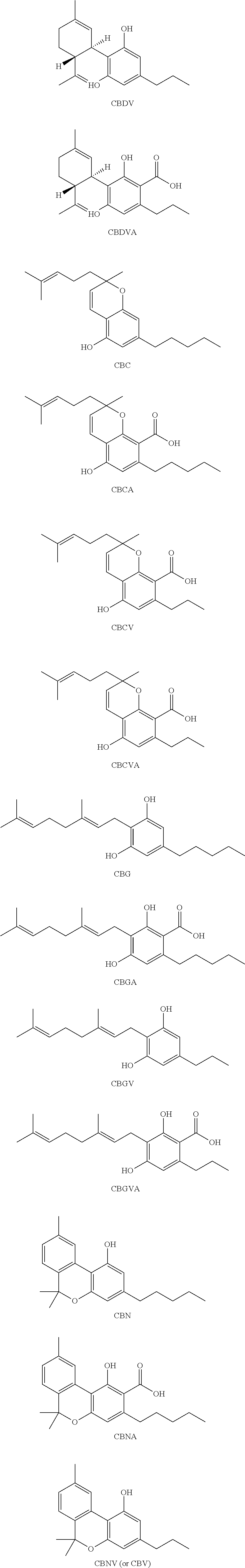

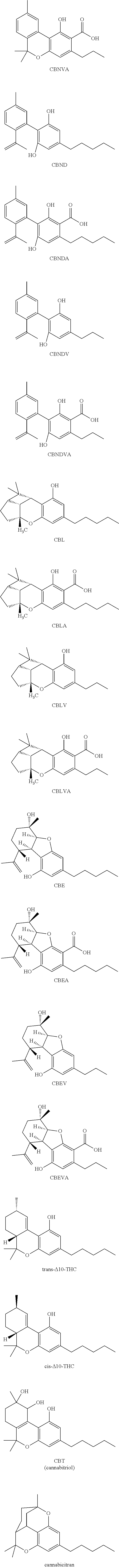

In select embodiments, the present disclosure relates to a method for converting cannabichromene (CBC) or a CBC derivative to cannabicyclol (CBL) or a CBL derivative, the method comprising contacting the CBC or CBC derivative with an acidic heterogeneous material to form a product mixture comprising the CBL or CBL derivative.

In the context of the present disclosure, the term “contacting” and its derivatives is intended to refer to bringing the CBC or CBC derivative and the acidic heterogeneous material into proximity such that a chemical reaction can occur. In some embodiments of the present disclosure, the contacting may be by adding the acidic heterogeneous material to the CBC or CBC derivative. In some embodiments, the contacting may be by combining, mixing, or both.

In the context of the present disclosure, the term “convert” includes a reaction involving a reagent initially present as an isolate, a component of a mixture (including a cannabis extract or distillate), and/or a reagent formed in situ.

In the context of the present disclosure, the term “CBC” refers to cannabichromene or, more generally, to cannabichromene-type cannabinoids. Accordingly the term “CBC” includes: (i) acid forms, such as “A-type”, “B-type”, or “AB-type” acid forms; (ii) salts of such acid forms, such as Na⁺ and/or Ca²⁺ salts of such acid forms; (iii) ester forms, such as those formed by hydroxyl-group esterification to form traditional esters, sulphonate esters, and/or phosphate esters; and/or (iv) various stereoisomers. In select embodiments of the present disclosure, CBC may have the following structural formula:

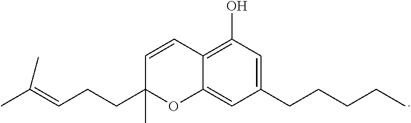 [SMILES: CCCCCc1cc(O)c2c(c1)OC(C)(CCC=C(C)C)C=C2]

In the context of the present disclosure, the term “CBL” refers to cannabicyclol or, more generally, to cannabicyclol-type cannabinoids. Accordingly the term “CBL” includes: (i) acid forms, such as “A-type”, “B-type”, or “AB-type” acid forms; (ii) salts of such acid forms, such as Na⁺ and/or Ca²⁺ salts of such acid forms; (iii) ester forms, such as those formed by hydroxyl-group esterification to form traditional esters, sulphonate esters, and/or phosphate esters; and/or (iv) various stereoisomers. In select embodiments of the present disclosure, CBL may have the following structural formula:

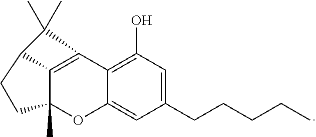

In the context of the present disclosure, an acid heterogeneous reagent is one which: (i) comprises one or more sites that are capable of accepting an electron pair from an electron pair donor or donating a proton to a proton-acceptor. A person of skill in the art will appreciate that acidic heterogeneous catalysts may thereby be Lewis-acidic or Bronsted/Lowry acidic, where Bronsted-Lowry acids are also Lewis acids. Importantly, the term “reagent” is used in the present disclosure to encompass both reactant-type reactivity (i.e. wherein the reagent is at least partly consumed as reactant is converted to product) and catalyst-type reactivity (i.e. wherein the reagent is not substantially consumed as reactant is converted to product). The acidity of the acidic heterogeneous reagent may be determined using Hammet indicators or temperature-programmed desorption (TPD). Acidity determined by TPD may more accurately reflect the number of acid sites able to react with reagents that are not small molecules.

Non-exclusive examples of acidic heterogeneous material include: montmorillonite, activated montmorillonite, amberlyst resins, and ZSM-5. In select embodiments of the present disclosure, the acidic heterogeneous material is activated montmorillonite.

In select embodiments of the present disclosure, the contacting is performed in the presence of a solvent (e.g. the reaction conditions comprise the components in a solvent). The solvent may dissolve at least a portion of the CBC or CBC derivative and/or a portion of the CBL or CBL derivative. Dissolving the CBC and/or the CBL (or derivatives thereof) may increase the rate of reaction. Without being bound to any particular theory, the present disclosure asserts that the properties of the solvent, including the polarity of the solvent, may affect the rate of reaction, the yield of the reaction, and/or the amount of side products formed. In select embodiments of the present disclosure, the solvent is chloroform. In select embodiments of the present disclosure, the solvent is a class 3 solvent. Any suitable class 3 solvent may be used. In select embodiments of the present disclosure, the class 3 solvent is heptane, acetic acid, or a combination thereof.

In select embodiments of the present disclosure, the contacting is at a reaction temperature of less than about 100° C. In select embodiments of the present disclosure, the reaction temperature is between about −40° C. and about 100° C., more particularly between about -25° C. and about 75° C., between about −20° C. and about 60° C., or between about −10° C. and about 10° C. In an embodiment, the reaction temperature is at about room temperature. In an embodiment, the reaction temperature is about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., or about 60° C. A higher temperature may increase the rate of reaction, where a higher rate of reaction increases throughput and is thereby more compatible with large scale reactions. A lower temperature may increase the yield of the reaction or reduce the amount of side products formed.

In select embodiments of the present disclosure, the contacting is for a reaction time of at least about 30 minutes, more particularly at least about 60 minutes. In an embodiment, the reaction time is between about 30 minutes and about 72 hours, more particularly between about 2 hours and about 48 hours, between about 4 hours and about 36 hours, between about 6 hours and about 24 hours, or between about 12 hours and about 24 hours. In an embodiment, the reaction time is between about 2 hours and 24 hours. In an embodiment, the reaction time is between about 18 hours and about 24 hours. In an embodiment, the reaction time is about 30 minutes, about 60 minutes, about 90 minutes, about 2 hours, about 4 hours, about 6 hours, about 8 hours, about 10 hours, about 12 hours, about 18 hours, about 24 hours, about 30 hours, about 36 hours, about 42 hours, about 48 hours, or longer. A shorter reaction time may increase the throughput of the reaction, which increases the rate at which CBL or CBL derivative is produced. A longer reaction time may increase the yield of the reaction.

In select embodiments of the present disclosure, the contacting is under a reaction pressure below about 1 bar (below about 100 kPa). In an embodiment, the reaction pressure is between about 0.1 mbar and 1000 mbar, more particularly between about 0.1 mbar and 100 mbar, or between about 0.5 mbar and 10 mbar. Executing the reaction under conditions of reduced pressure may reduce the amount of side products present in a product of the reaction. Non-exclusive examples of side products include CBC, tetrahydrocannabinol (THC), cannabacitran, cannabigerol (CBG), cannabidiol (CBD), and cannabinol (CBN).

In select embodiments of the present disclosure, the product mixture comprises at least about 1% w/w CBL and at least about 1% w/w CBC (or derivatives thereof).

In select embodiments of the present disclosure, the product mixture comprises between about 1% w/w CBC and about 95% w/w CBC, or more particularly between about 1% w/w CBC and about 75% w/w CBC, between about 1% w/w CBC and about 50% w/w CBC, or between about 1% w/w CBC and about 25% w/w CBC. In an embodiment, the product mixture comprises between about 1% w/w CBC and about 25% w/w CBC, more particularly between about 1% w/w CBC and about 25% w/w CBC, or between about 1% w/w CBC and about 5% w/w CBC. In an embodiment, the product mixture comprises between about 5% w/w CBC and about 25% w/w CBC. In an embodiment, the product mixture comprises about 1.0%, about 2.5%, about 5.0%, about 7.5%, about 10.0%, about 12.5%, about 15.0%, about 17.5%, about 20.0%, about 22.5%, about 25%, about 27.5%, about 30%, or greater w/w CBC. As will be appreciated from the disclosure herein, reference to CBC in this paragraph equally applies to CBC derivatives and the amounts thereof.

In select embodiments of the present disclosure, the product mixture comprises between about 1% w/w CBL and about 99% w/w CBL, more particularly between about 10% w/w CBL and about 90% w/w CBL, between about 25% w/w CBL and about 75% w/w CBL or between about 40% w/w CBL and about 50% CBL. In an embodiment, the product mixture comprises about 1.0%, about 2.5%, about 5.0%, about 7.5%, about 10.0%, about 12.5%, about 15.0%, about 17.5%, about 20.0%, about 22.5%, about 25%, about 27.5%, about 30%, about 32.5%, about 35%, about 37.5%, about 40%, about 42.5%, about 45%, about 47.5%, about 50%, or greater w/w CBL. As will be appreciated from the disclosure herein, reference to CBL in this paragraph equally applies to CBL derivatives and the amounts thereof.

In the context of the present disclosure, the relative quantities of CBL and CBC in a particular composition may be expressed as a ratio—CBL:CBC. Those skilled in the art will recognize that a variety of analytical methods may be used to determine such ratios, and the protocols required to implement any such method are within the purview of those skilled in the art. By way of non-limiting example, CBL:CBC ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof. As will be appreciated from the disclosure herein, reference to CBL:CBC ratios may be equally applicable to ratios of derivatives of these compounds.

In select embodiments of the present disclosure, the CBL and the CBC are present in a CBL:CBC ratio that is between about 1000:1 and about 1:1000, between about 500:1 and about 1:500, between about 100:1 and about 1:100, between about 10:1 and about 1:10, between about 5:1 and about 1:5, or between about 2:1 and about 1:2. In an embodiment, the CBL:CBC ratio of the product mixture is between about 100:1 and about 1:20, between about 95:1 and about 1:10, or between about 20:1 and about 1:1. In an embodiment, the CBL:CBC ratio of the product mixture is about 20:1, about 15:1, about 10:1, about 5:1, about 4:1, about 3:1, about 2:1, about 1:1, about 1:2, about 1:3, about 1:4, about 1:5, about 1:10, about 1:15, or about 1:20.

In select embodiments of the present disclosure, the CBL derivative of the form:

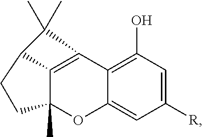

wherein R is methyl, propyl, heptyl, 1,1-dimethylheptyl, phenylethyl, or phenylvinyl. A CBL derivative may possess enhanced or modified medicinal and/or recreational effect, and may provide additional functionality for synthetic chemists.

In select embodiments of the present disclosure, the CBC derivative of the form:

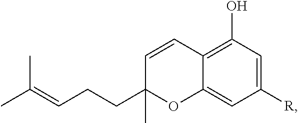

wherein R is methyl, propyl, heptyl, 1,1-dimethylheptyl, phenylethyl, or phenylvinyl. As will be appreciated by those skilled in the art who have benefitted from the teachings of the present disclosure, a derivative of CBL converted from a CBC derivative will typically share the same R group.

In select embodiments of the present disclosure, the method further comprises purifying the product mixture to provide CBL or CBL derivative that is at least 50%, at least 75%, at least 80%, at least 85%, at least 90%, or at least 95% pure. In an embodiment, the CBL or CBL derivative is at least 95% pure. CBL or CBL derivative with such purity may be more readily used as an API. Non-exclusive examples of purification methods include chromatography, flash chromatography, reversed phase C18 flash chromatography, simulated moving bed chromatography, liquid-liquid extraction, distillation, short-path distillation, and crystallization. Chromatography methods may be surprisingly effective for the isolation of CBL or CBL derivative from CBC or CBC derivative. In select embodiments of the present disclosure, purifying the product mixture comprises flash chromatography. In select embodiments of the present disclosure, the flash chromatography is reversed phase C18 flash chromatography. Flash chromatography and/or reversed phase C18 flash chromatography may be particularly effective for purifying CBL or CBL derivative. In select embodiments, purifying the product mixture includes crystallizing the product mixture to produce CBL or CBL derivative that has a purity of at least 95%, at least 99%, or greater than 99%. Crystalline CBL or CBL derivative that has a purity of at least 95% may be a CBL isolate or CBL derivative isolate.

In select embodiments of the present disclosure, the CBC or CBC derivative is a component of a cannabis extract, a cannabis distillate, a cannabis isolate, or a cannabis synthesis reaction mixture. As the CBC derivative may not be a naturally occurring substance, in particular embodiments the CBC derivative is a component of a cannabis synthesis reaction mixture. In the context of the present disclosure, a cannabis synthesis reaction mixture comprises a reaction mixture comprising the products of a reaction between citral and a modified resorcinol. In select embodiments of the present disclosure, the cannabis extract, the cannabis distillate, the cannabis isolate, or the cannabis synthesis reaction mixture comprises at least 40% w/w CBC or CBC derivative. In select embodiments of the present disclosure, the cannabis extract, the cannabis distillate, the cannabis isolate, or the cannabis synthesis reaction mixture comprises between about 40% w/w and about 95% w/w, between about 50% w/w and about 95% w/w, or between about 85% w/w and about 90% w/w CBC or CBC derivative. The methods of the present disclosure allow conversion of CBC to CBL (or derivatives thereof) where the CBC or CBC derivative is a component of a mixture (e.g. a cannabis extract, a cannabis distillate, a cannabis isolate, or a cannabis synthesis reaction mixture), which allows the use of reagents with reduced CBC or CBC derivative purity that may be more readily available or less expensive than reagents of higher purity.

In select embodiments of the present disclosure, the CBL or CBL derivative has a yield of at least 10%. In some embodiments, the yield of CBL or CBL derivative may be at least 25%, at least 50%, or more. A higher yield increases the amount of CBL or CBL derivatives obtained via the methods of the present disclosure.

In select embodiments of the present disclosure, the product mixture comprises less than about 30% w/w tetrahydrocannabinol (THC). In an embodiment, the product mixture comprises less than 30% w/w THC, less than 25% w/w THC, less than 20% w/w THC, less than 15% w/w THC, less than 10% w/w THC, less than 5% w/w THC, less than 2.5% w/w THC, less than 1% w/w THC, less than 0.5% w/w THC, less than 0.3% w/w THC, less than 0.1% w/w THC, or even less. In an embodiment, the product mixture comprises only trace amounts of THC (e.g. less than 0.01% w/w THC). In an embodiment, the product mixture comprises no THC or at least no detectable THC. In the context of the present disclosure, the phrase “less than” includes amounts below the detection limit of appropriate analytical methods.

A lower amount of THC may allow a product mixture to avoid regulatory restrictions. In select embodiments of the present disclosure, a CBL:THC ratio of the product mixture is between about 10,000:1 and about 1:1, between about 1000:1 and about 1:1, between about 500:1 and about 1:1, between about 100:1 and about 1:1, or between about 10:1 and about 1:1. In an embodiment, the CBL:THC ratio is between about 100:1 and about 10:1, between about 100:1 and about 25:1, between about 100:1 and about 50:1, or between about 100:1 and about 75:1. As will be appreciated from the disclosure herein, reference to CBL in this paragraph equally applies to CBL derivatives.

In select embodiments of the present disclosure, the product mixture comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a CBL:CBD ratio of the product mixture may be between about 100:1 and about 1:100, more particularly between about 100:1 and about 1:10. In select embodiments of the present disclosure, the CBL:CBD ratio of the product mixture may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the CBL:CBD ratio of the product mixture may be about 10:1, about 5:1, about 2:1, about 1:1, about 1:2, about 1:5 or about 1:10. As will be appreciated from the disclosure herein, reference to CBL in this paragraph equally applies to CBL derivatives.

In select embodiments, the present disclosure relates to a method for converting cannabichromene (CBC) or a CBC derivative to cannabicyclol (CBL) or a CBL derivative, the method comprising contacting the CBC or CBC derivative with a radical initiator to form a product mixture comprising the CBL or CBL derivative.

In the context of the present disclosure, the term “contacting” and its derivatives is intended to refer to bringing the CBC or CBC derivative and the radical initiator into proximity such that a chemical reaction can occur. In some embodiments of the present disclosure, the contacting may be by adding the radical initiator to the CBC or CBC derivative. In some embodiments, the contacting may be by combining, mixing, or both.

In the context of the present disclosure, a radical initiator is a source of free radicals. The radical initiator may generate free radicals under the conditions disclosed herein at a higher rate than under ambient conditions. Non-exclusive examples of free radical initiators include metal ions and organic compounds. Non-exclusive examples of metal ion free radical initiators include transition metal ions (e.g. Cu²⁺, Fe³⁺), their corresponding complexes, and their corresponding salts. Non-exclusive examples of organic free radical initiators include organic peroxides. In select embodiments of the present disclosure, the radical initiator is Fe(ClO₄)₃.

In select embodiments of the present disclosure, the contacting of the CBC or CBC derivative with the radical initiator is performed in the presence of a solvent. The solvent may dissolve at least a portion of the CBC or CBC derivative and/or a portion of the formed CBL or CBL derivative. Dissolving the CBC and/or the CBL (or derivatives thereof) may increase the rate of reaction. Without being bound to any particular theory, the present disclosure asserts that the properties of the solvent, including the polarity of the solvent, may affect the rate of reaction, the yield of the reaction, and/or the amount of side products formed. In select embodiments of the present disclosure, the solvent is a class 3 solvent. Any suitable class 3 solvent may be used. In select embodiments of the present disclosure, the solvent is ethyl acetate.

In select embodiments of the present disclosure, the contacting with the radical initiator is at a reaction temperature of less than about 77° C., more particularly between about 0° C. and about 77° C., between about 10° C. and about 50° C., or between about 15° C. and about 25° C. In an embodiment, the reaction temperature is about 0° C., about 5° C., about 15° C., about 20° C., about 25° C., about 30° C., about 35° C., about 40° C., about 45° C., or about 50° C.

In select embodiments of the present disclosure, the contacting with the radical initiator is for a reaction time of at least about 1 minute, more particularly between about 1 minute and about 60 minutes, between about 5 minutes and about 60 minutes, between about 10 minutes and about 30 minutes, or between about 10 minutes and about 20 minutes. In an embodiment, the reaction time is about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or longer.

In select embodiments of the present disclosure, the contacting with the radical initiator is under a reaction pressure below about 1 bar (below about 100 kPa). In an embodiment, the reaction pressure is between about 0.1 mbar and 1000 mbar, more particularly between about 0.1 mbar and 100 mbar, or between about 0.5 mbar and 10 mbar.

In select embodiments of the present disclosure, the product mixture comprises at least about 1% w/w CBL and at least about 1% w/w CBC (or derivatives thereof).

In select embodiments of the present disclosure, the product mixture comprises between about 1% w/w CBC and about 95% w/w CBC, or more particularly between about 1% w/w CBC and about 75% w/w CBC, between about 1% w/w CBC and about 50% w/w CBC, or between about 1% w/w CBC and about 25% w/w CBC. In an embodiment, the product mixture comprises between about 1% w/w CBC and about 25% w/w CBC, more particularly between about 1% w/w CBC and about 25% w/w CBC, or between about 1% w/w CBC and about 5% w/w CBC. In an embodiment, the product mixture comprises between about 5% w/w CBC and about 25% w/w CBC. In an embodiment, the product mixture comprises about 1.0%, about 2.5%, about 5.0%, about 7.5%, about 10.0%, about 12.5%, about 15.0%, about 17.5%, about 20.0%, about 22.5%, about 25%, about 27.5%, about 30%, or greater w/w CBC. As will be appreciated from the disclosure herein, reference to CBC in this paragraph equally applies to CBC derivatives and the amounts thereof.

In select embodiments of the present disclosure, the product mixture comprises between about 1% w/w CBL and about 99% w/w CBL, more particularly between about 10% w/w CBL and about 90% w/w CBL, between about 25% w/w CBL and about 75% w/w CBL or between about 40% w/w CBL and about 50% CBL. In an embodiment, the product mixture comprises about 1.0%, about 2.5%, about 5.0%, about 7.5%, about 10.0%, about 12.5%, about 15.0%, about 17.5%, about 20.0%, about 22.5%, about 25%, about 27.5%, about 30%, about 32.5%, about 35%, about 37.5%, about 40%, about 42.5%, about 45%, about 47.5%, about 50%, or greater w/w CBL. As will be appreciated from the disclosure herein, reference to CBL in this paragraph equally applies to CBL derivatives and the amounts thereof.

In select embodiments of the present disclosure, the CBL and the CBC are present in a CBL:CBC ratio that is between about 1000:1 and about 1:1000, between about 500:1 and about 1:500, between about 100:1 and about 1:100, between about 10:1 and about 1:10, between about 5:1 and about 1:5, or between about 2:1 and about 1:2. In an embodiment, the CBL:CBC ratio of the product mixture is between about 100:1 and about 1:20, between about 95:1 and about 1:10, or between about 20:1 and about 1:1. In an embodiment, the CBL:CBC ratio of the product mixture is about 20:1, about 15:1, about 10:1, about 5:1, about 4:1, about 3:1, about 2:1, about 1:1, about 1:2, about 1:3, about 1:4, about 1:5, about 1:10, about 1:15, or about 1:20. As will be appreciated from the disclosure herein, reference to CBL:CBC ratios may be equally applicable to ratios of derivatives of these compounds.

In select embodiments of the present disclosure, the CBL derivative is of the form:

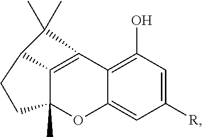

wherein R is methyl, propyl, heptyl, 1,1-dimethylheptyl, phenylethyl, or phenylvinyl.

In select embodiments of the present disclosure, the CBC derivative is of the form:

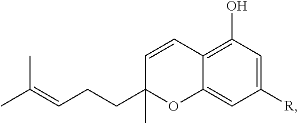

wherein R is methyl, propyl, heptyl, 1,1-dimethylheptyl, phenylethyl, or phenylvinyl. As will be appreciated by those skilled in the art who have benefitted from the teachings of the present disclosure, a derivative of CBL converted from a CBC derivative will typically share the same R group.

In select embodiments of the present disclosure, the method further comprises purifying the product mixture to provide CBL or CBL derivative that is at least 50%, at least 75%, at least 80%, at least 85%, at least 90%, or at least 95% pure. In an embodiment, the CBL or CBL derivative is at least 95% pure. In select embodiments of the present disclosure, purifying the product mixture comprises flash chromatography. In select embodiments of the present disclosure, the flash chromatography is reversed phase C18 flash chromatography. In select embodiments, purifying the product mixture includes crystallizing the product mixture to produce CBL or CBL derivative that has a purity of at least 95%, at least 99%, or greater than 99%. Crystalline CBL or CBL derivative that has a purity of at least 95% may be a CBL isolate or CBL derivative isolate.

In select embodiments of the present disclosure, the CBC or CBC derivative is a component of a cannabis extract, a cannabis distillate, a cannabis isolate, or a cannabis synthesis reaction mixture. As the CBC derivative may not be a naturally occurring substance, in particular embodiments the CBC derivative is a component of a cannabis synthesis reaction mixture. In select embodiments of the present disclosure, the cannabis extract, distillate, or isolate comprises at least 40% w/w CBC or CBC derivative. In select embodiments of the present disclosure, the cannabis extract, the cannabis distillate, the cannabis isolate, or the cannabis synthesis reaction mixture comprises between about 40% w/w and about 95% w/w, between about 50% w/w and about 95% w/w, or between about 85% w/w and about 90% w/w CBC or CBC derivative.

In select embodiments of the present disclosure, the CBL or CBL derivative has a yield of at least 10%. In some embodiments, the yield of CBL or CBL derivative may be at least 25%, at least 50%, or more. In select embodiments of the methods of the present disclosure involving contacting with a radical initiator, the concentration of CBC or CBC derivative prior to contacting the CBC or CBC derivative with the radical initiator is between 0.1 mM and 10 mM, and the concentration of the radical initiator prior to contacting the CBC or CBC derivative is between 0.01 mM and 1 mM.

In select embodiments of the present disclosure, the product mixture comprises less than about 30% w/w tetrahydrocannabinol (THC). In an embodiment, the product mixture comprises less than 30% w/w THC, less than 25% w/w THC, less than 20% w/w THC, less than 15% w/w THC, less than 10% w/w THC, less than 5% w/w THC, less than 2.5% w/w THC, less than 1% w/w THC, less than 0.5% w/w THC, less than 0.3% w/w THC, less than 0.1% w/w THC, or even less. In an embodiment, the product mixture comprises only trace amounts of THC (e.g. less than 0.01% w/w THC). In an embodiment, the product mixture comprises no THC or at least no detectable THC.

In select embodiments of the present disclosure, a CBL:THC ratio of the product mixture is between about 10,000:1 and about 1:1, between about 1000:1 and about 1:1, between about 500:1 and about 1:1, between about 100:1 and about 1:1, or between about 10:1 and about 1:1. In an embodiment, the CBL:THC ratio is between about 100:1 and about 10:1, between about 100:1 and about 25:1, between about 100:1 and about 50:1, or between about 100:1 and about 75:1. As will be appreciated from the disclosure herein, reference to CBL in this paragraph equally applies to CBL derivatives.

In select embodiments of the present disclosure, the product mixture comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a CBL:CBD ratio of the product mixture may be between about 100:1 and about 1:100, more particularly between about 100:1 and about 1:10. In select embodiments of the present disclosure, the CBL:CBD ratio of the product mixture may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the CBL:CBD ratio of the product mixture may be about 10:1, about 5:1, about 2:1, about 1:1, about 1:2, about 1:5 or about 1:10. As will be appreciated from the disclosure herein, reference to CBL in this paragraph equally applies to CBL derivatives.

In select embodiments, the present disclosure relates to a method for converting CBC or a CBC derivative to CBL or a CBL derivative, the method comprising irradiating the CBC or CBC derivative with UV light to form a product mixture comprising CBL or CBL derivative.

In the context of the present disclosure, irradiating comprises contacting with UV light. The UV light may be of a single wavelength, multiple wavelengths, or a spectrum of wavelengths. The UV light may be a portion of an irradiating light. In an embodiment, the UV light is of a wavelength between 100 nm and 400 nm. In an embodiment, the UV light is of a wavelength between 300 nm and 350 nm.

In select embodiments of the present disclosure, the irradiating the CBC or CBC derivative is performed under inert atmosphere. Irradiating under an inert atmosphere may reduce the formation of side products.

In select embodiments of the present disclosure, the irradiating is performed in the presence of a solvent. The solvent may dissolve at least a portion of the CBC or CBC derivative and/or a portion of the CBL or CBL derivative. In select embodiments of the present disclosure, the solvent is a class 3 solvent. Any suitable class 3 solvent may be used. In select embodiments of the present disclosure, the solvent is a 1:1 v/v mixture of isopropyl alcohol and acetone. In select embodiments of the present disclosure, the solvent is acetone.

In select embodiments of the present disclosure, the irradiating is at a reaction temperature of less than about 60° C. In select embodiments of the present disclosure, the reaction temperature is between about 10° C. and about 60° C., more particularly between about 10° C. and about 50° C., between about 15° C. and about 35° C., or between about 15° C. and about 25° C. In an embodiment, the reaction temperature is at about room temperature. In an embodiment, the reaction temperature is about 10° C., about 15° C., about 20° C., about 25° C., about 30° C., about 35° C., about 40° C., about 45° C., about 50° C., about 55° C., or about 60° C. A higher temperature may increase the rate of reaction, where a higher rate of reaction increases throughput and is thereby more compatible with large scale reactions. A lower temperature may increase the yield of the reaction or reduce the amount of side products formed.

In select embodiments of the present disclosure, the irradiating is for a reaction time of at least about 30 minutes, more particularly at least about 60 minutes. In an embodiment, the reaction time is between about 30 minutes and about 7 days, more particularly between about 2 hours and about 7 days, between about 2 hours and about 48 hours, between about 2 hours and about 12 hours, or between about 2 hours and about 6 hours. In an embodiment, the reaction time is about 30 minutes, about 60 minutes, about 90 minutes, about 2 hours, about 4 hours, about 6 hours, about 8 hours, about 10 hours, about 12 hours, about 18 hours, about 24 hours, about 30 hours, about 36 hours, about 42 hours, about 48 hours, or longer. A shorter reaction time may increase the throughput of the reaction, which increases the rate at which CBL is produced. A longer reaction time may increase the yield of the reaction.

In select embodiments of the present disclosure, the irradiating is under a reaction pressure below about 1 bar (below about 100 kPa). In an embodiment, the reaction pressure is between about 0.1 mbar and 1000 mbar, more particularly between about 0.1 mbar and 100 mbar, or between about 0.5 mbar and 10 mbar.

In select embodiments of the present disclosure, the product mixture comprises at least about 1% w/w CBL and at least about 1% w/w CBC (or derivatives thereof).

In select embodiments of the present disclosure, the product mixture comprises between about 1% w/w CBC and about 95% w/w CBC, or more particularly between about 1% w/w CBC and about 75% w/w CBC, between about 1% w/w CBC and about 50% w/w CBC, or between about 1% w/w CBC and about 25% w/w CBC. In an embodiment, the product mixture comprises between about 1% w/w CBC and about 25% w/w CBC, more particularly between about 1% w/w CBC and about 25% w/w CBC, or between about 1% w/w CBC and about 5% w/w CBC. In an embodiment, the product mixture comprises between about 5% w/w CBC and about 25% w/w CBC. In an embodiment, the product mixture comprises about 1.0%, about 2.5%, about 5.0%, about 7.5%, about 10.0%, about 12.5%, about 15.0%, about 17.5%, about 20.0%, about 22.5%, about 25%, about 27.5%, about 30%, or greater w/w CBC. As will be appreciated from the disclosure herein, reference to CBC in this paragraph equally applies to CBC derivatives and the amounts thereof.

In select embodiments of the present disclosure, the product mixture comprises between about 1% w/w CBL and about 99% w/w CBL, more particularly between about 10% w/w CBL and about 90% w/w CBL, between about 25% w/w CBL and about 75% w/w CBL or between about 40% w/w CBL and about 50% CBL. In an embodiment, the product mixture comprises about 1.0%, about 2.5%, about 5.0%, about 7.5%, about 10.0%, about 12.5%, about 15.0%, about 17.5%, about 20.0%, about 22.5%, about 25%, about 27.5%, about 30%, about 32.5%, about 35%, about 37.5%, about 40%, about 42.5%, about 45%, about 47.5%, about 50%, or greater w/w CBL. As will be appreciated from the disclosure herein, reference to CBL in this paragraph equally applies to CBL derivatives and the amounts thereof.

In select embodiments of the present disclosure, the CBL and the CBC are present in a CBL:CBC ratio that is between about 1000:1 and about 1:1000, between about 500:1 and about 1:500, between about 100:1 and about 1:100, between about 10:1 and about 1:10, between about 5:1 and about 1:5, or between about 2:1 and about 1:2. In an embodiment, the CBL:CBC ratio of the product mixture is between about 100:1 and about 1:20, between about 95:1 and about 1:10, or between about 20:1 and about 1:1. In an embodiment, the CBL:CBC ratio of the product mixture is about 20:1, about 15:1, about 10:1, about 5:1, about 4:1, about 3:1, about 2:1, about 1:1, about 1:2, about 1:3, about 1:4, about 1:5, about 1:10, about 1:15, or about 1:20. As will be appreciated from the disclosure herein, reference to CBL:CBC ratios may be equally applicable to ratios of derivatives of these compounds.

In select embodiments of the present disclosure, the CBL derivative of the form:

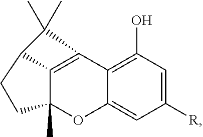

wherein R is methyl, propyl, heptyl, 1,1-dimethylheptyl, phenylethyl, or phenylvinyl.

In select embodiments of the present disclosure, the CBC derivative of the form:

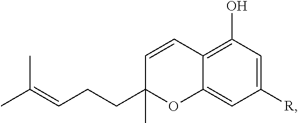

wherein R is methyl, propyl, heptyl, 1,1-dimethylheptyl, phenylethyl, or phenylvinyl. As will be appreciated by those skilled in the art who have benefitted from the teachings of the present disclosure, a derivative of CBL converted from a CBC derivative will typically share the same R group.

In select embodiments of the present disclosure, the method further comprises purifying the product mixture to provide CBL or CBL derivative that is at least 50%, at least 75%, at least 80%, at least 85%, at least 90%, or at least 95% pure. In an embodiment, the CBL or CBL derivative is at least 95% pure. In select embodiments of the present disclosure, purifying the product mixture comprises flash chromatography. In select embodiments of the present disclosure, the flash chromatography is reversed phase C18 flash chromatography. In select embodiments, purifying the product mixture includes crystallizing the product mixture to produce CBL or CBL derivative that has a purity of at least 95%, at least 99%, or greater than 99%. Crystalline CBL or CBL derivative that has a purity of at least 95% may be a CBL isolate or CBL derivative isolate.

In select embodiments of the present disclosure, the CBC or CBC derivative is a component of a cannabis extract, a cannabis distillate, a cannabis isolate, or a cannabis synthesis reaction mixture. As the CBC derivative may not be a naturally occurring substance, in particular embodiments the CBC derivative is a component of a cannabis synthesis reaction mixture. In select embodiments of the present disclosure, the cannabis extract, the cannabis distillate, the cannabis isolate, or the cannabis synthesis reaction mixture comprises at least 40% w/w CBC or CBC derivative. In select embodiments of the present disclosure, the cannabis extract, the cannabis distillate, the cannabis isolate, or the cannabis synthesis reaction mixture comprises between about 40% w/w and about 95% w/w, between about 50% w/w and about 95% w/w, or between about 85% w/w and about 90% w/w CBC or CBC derivative.

In select embodiments of the present disclosure, the CBL or CBL derivative has a yield of at least 10%. In some embodiments, the yield of CBL or CBL derivative may be at least 25%, at least 50%, or more. A higher yield increases the amount of CBL or CBL derivatives obtained via the methods of the present disclosure.

In select embodiments of the present disclosure, a wavelength of the UV light is between 300 nm and 350 nm. In select embodiments of the present disclosure, the UV light has a power between 30 W and 500 W. In the context of the present disclosure, the power of the UV light is defined as the electrical power consumed by the light source in W.

In select embodiments of the present disclosure, the product mixture comprises less than about 30% w/w tetrahydrocannabinol (THC). In an embodiment, the product mixture comprises less than 30% w/w THC, less than 25% w/w THC, less than 20% w/w THC, less than 15% w/w THC, less than 10% w/w THC, less than 5% w/w THC, less than 2.5% w/w THC, less than 1% w/w THC, less than 0.5% w/w THC, less than 0.3% w/w THC, less than 0.1% w/w THC, or even less. In an embodiment, the product mixture comprises only trace amounts of THC (e.g. less than 0.01% w/w THC). In an embodiment, the product mixture comprises no THC or at least no detectable THC.

In select embodiments of the present disclosure, a CBL:THC ratio of the product mixture is between about 10,000:1 and about 1:1, between about 1000:1 and about 1:1, between about 500:1 and about 1:1, between about 100:1 and about 1:1, or between about 10:1 and about 1:1. In an embodiment, the CBL:THC ratio is between about 100:1 and about 10:1, between about 100:1 and about 25:1, between about 100:1 and about 50:1, or between about 100:1 and about 75:1. As will be appreciated from the disclosure herein, reference to CBL in this paragraph equally applies to CBL derivatives.

In select embodiments of the present disclosure, the product mixture comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a CBL:CBD ratio of the product mixture may be between about 100:1 and about 1:100, more particularly between about 100:1 and about 1:10. In select embodiments of the present disclosure, the CBL:CBD ratio of the product mixture may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the CBL:CBD ratio of the product mixture may be about 10:1, about 5:1, about 2:1, about 1:1, about 1:2, about 1:5 or about 1:10. As will be appreciated from the disclosure herein, reference to CBL in this paragraph equally applies to CBL derivatives.

In select embodiments, the present disclosure relates to a cannabinoid composition comprising (i) at least about 4% w/w CBL or a CBL derivative and (ii) at least about 4% w/w CBC or a CBC derivative. The cannabinoid composition may, for example, be prepared by any of the methods disclosed herein.

In select embodiments of the present disclosure, the cannabinoid composition comprises between about 4% w/w CBL and about 99% w/w CBL, between about 10% w/w CBL and about 90% w/w CBL, between about 25% w/w CBL and about 75% w/w CBL or between about 40% w/w CBL and about 50% CBL. In select embodiments of the present disclosure, the cannabinoid composition comprises about 5%, about 15%, about 25%, about 35%, about 50%, about 60%, about 75%, about 85%, about 90%, or about 95% w/w CBL. In select embodiments of the present disclosure, the cannabinoid composition comprises between 4% w/w CBC and about 10% w/w CBC, between about 10% w/w CBC and about 25% w/w CBC, or greater than about 25% w/w CBC. In select embodiments of the present disclosure, the cannabinoid composition comprises between 40% w/w CBC and about 50% w/w CBC, between about 50% w/w CBC and about 70% w/w CBC, or greater than about 70% w/w CBC. In select embodiments of the present disclosure, the cannabinoid composition comprises about 40%, about 50%, about 60%, about 70%, about 80%, about 90%, or about 95% w/w CBC. As will be appreciated from the disclosure herein, reference to CBL and CBC in this paragraph equally applies to CBL derivatives and CBC derivatives, respectively, and the amounts thereof.

In select embodiments of the present disclosure, the CBL and the CBC are present in a CBL:CBC ratio that is between about 1000:1 and about 1:1000, between about 500:1 and about 1:500, between about 100:1 and about 1:100, between about 10:1 and about 1:10, between about 5:1 and about 1:5, or between about 2:1 and about 1:2. In an embodiment, the CBL:CBC ratio of the cannabinoid composition is between about 100:1 and about 1:20, between about 95:1 and about 1:10, or between about 20:1 and about 1:1. In an embodiment, the CBL:CBC ratio of the cannabinoid composition is about 20:1, about 15:1, about 10:1, about 5:1, about 4:1, about 3:1, about 2:1, about 1:1, about 1:2, about 1:3, about 1:4, about 1:5, about 1:10, about 1:15, or about 1:20. As will be appreciated from the disclosure herein, reference to CBL:CBC ratios may be equally applicable to ratios of derivatives of these compounds.

In select embodiments of the present disclosure, the cannabinoid composition comprises a CBL derivative of the form:

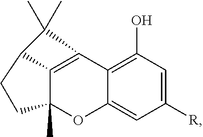

and comprises a CBC derivative of the form:

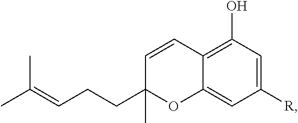

wherein R is methyl, propyl, heptyl, 1,1-dimethylheptyl, phenylethyl, or phenylvinyl. As will be appreciated by those skilled in the art who have benefitted from the teachings of the present disclosure, the CBL derivative and CBC derivative in the cannabinoid composition will typically share the same R group.

In select embodiments of the present disclosure, the cannabinoid composition comprises less than about 30% w/w tetrahydrocannabinol (THC). In an embodiment, the cannabinoid composition comprises less than 30% w/w THC, less than 25% w/w THC, less than 20% w/w THC, less than 15% w/w THC, less than 10% w/w THC, less than 5% w/w THC, less than 2.5% w/w THC, less than 1% w/w THC, less than 0.5% w/w THC, less than 0.3% w/w THC, less than 0.1% w/w THC, or even less. In an embodiment, the cannabinoid composition comprises only trace amounts of THC (e.g. less than 0.01% w/w THC). In an embodiment, the cannabinoid composition comprises no THC or at least no detectable THC.

In select embodiments of the present disclosure, a CBL:THC ratio of the cannabinoid composition is between about 1000:1 and about 1:1 or between about 100:1 and about 10:1. In select embodiments of the present disclosure, the CBL:THC ratio of the cannabinoid composition is between about 10,000:1 and about 1:1, between about 1000:1 and about 1:1, between about 500:1 and about 1:1, between about 100:1 and about 1:1, or between about 10:1 and about 1:1. In an embodiment, the CBL:THC ratio is between about 100:1 and about 10:1, between about 100:1 and about 25:1, between about 100:1 and about 50:1, or between about 100:1 and about 75:1. As will be appreciated from the disclosure herein, reference to CBL in this paragraph equally applies to CBL derivatives.

In select embodiments of the present disclosure, the cannabinoid composition comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a CBL:CBD ratio of the cannabinoid composition may be between about 100:1 and about 1:100, more particularly between about 100:1 and about 1:10. In select embodiments of the present disclosure, the CBL:CBD ratio of the cannabinoid composition may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the CBL:CBD ratio of the cannabinoid composition may be about 10:1, about 5:1, about 2:1, about 1:1, about 1:2, about 1:5 or about 1:10. As will be appreciated from the disclosure herein, reference to CBL in this paragraph equally applies to CBL derivatives.

In select embodiments, the present disclosure relates to a method of preparing CBL or a CBL derivative, the method comprising: heating a reaction mixture comprising citral, a modified resorcinol, and an amine to form a first product mixture; and converting the first product mixture into a second product mixture by: (i) contacting the first product mixture with an acidic heterogeneous material to form the CBL or the CBL derivative; (ii) contacting the first product mixture with a radical initiator to form the CBL or the CBL derivative; or (iii) irradiating the first product mixture with UV light to form the CBL or the CBL derivative. In select embodiments of the present disclosure, the first reaction mixture is a cannabis synthesis reaction mixture.

In select embodiments of the present disclosure, and in relation to methods for preparing CBL, the modified resorcinol may be of the form:

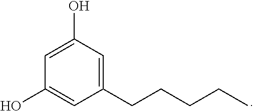

In select embodiments of the present disclosure, and in relation to methods for preparing CBL derivatives, the modified resorcinol may be of the form:

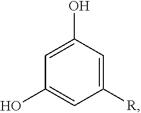

and the CBL derivative of the form:

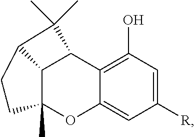

wherein R is methyl, propyl, heptyl, 1,1-dimethylheptyl, phenylethyl, or phenylvinyl. As will be appreciated by those skilled in the art who have benefitted from the teachings of the present disclosure, a derivative of CBL prepared from a modified resorcinol will typically share the same R group.

In select embodiments, the present disclosure relates to a method of producing an artificial resin, the method comprising: contacting a starting material comprising CBC or a CBC derivative with an acidic heterogeneous material to form an artificial resin comprising at least about 4% w/w CBL or CBL derivative, a first cannabinoid, and a second cannabinoid.

In select embodiments of the present disclosure, the acidic heterogeneous material converts CBC to CBL or CBC derivative to CBL derivative in the presence of at least one additional cannabinoid to produce the artificial resin. Unreacted CBC or CBC derivative may form a portion of the artificial resin.

Non-exclusive examples of acidic heterogeneous material include: montmorillonite, activated montmorillonite, amberlyst resins, and ZSM-5. In select embodiments of the present disclosure, the acidic heterogeneous material is activated montmorillonite.

In select embodiments of the present disclosure, the contacting is performed in the presence of a solvent. In select embodiments of the present disclosure, the solvent is chloroform. In select embodiments of the present disclosure, the solvent is a class 3 solvent. Any suitable class 3 solvent may be used. In select embodiments of the present disclosure, the class 3 solvent is heptane, acetic acid, or a combination thereof.

In select embodiments of the present disclosure, the contacting is at a reaction temperature of less than about 100° C. In select embodiments of the present disclosure, the reaction temperature is between about −40° C. and about 100° C., more particularly between about −25° C. and about 75° C., between about −20° C. and about 60° C., or between about −10° C. and about 10° C. In an embodiment, the reaction temperature is at about room temperature. In an embodiment, the reaction temperature is about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., or about 60° C.

In select embodiments of the present disclosure, the contacting is for a reaction time of at least about 30 minutes, more particularly at least about 60 minutes. In an embodiment, the reaction time is between about 30 minutes and about 72 hours, more particularly between about 2 hours and about 48 hours, between about 4 hours and about 36 hours, between about 6 hours and about 24 hours, or between about 12 hours and about 24 hours. In an embodiment, the reaction time is between about 2 hours and 24 hours. In an embodiment, the reaction time is between about 18 hours and about 24 hours. In an embodiment, the reaction time is about 30 minutes, about 60 minutes, about 90 minutes, about 2 hours, about 4 hours, about 6 hours, about 8 hours, about 10 hours, about 12 hours, about 18 hours, about 24 hours, about 30 hours, about 36 hours, about 42 hours, about 48 hours, or longer.

In select embodiments of the present disclosure, the contacting is under a reaction pressure below about 1 bar (below about 100 kPa). In an embodiment, the reaction pressure is between about 0.1 mbar and 1000 mbar, more particularly between about 0.1 mbar and 100 mbar, or between about 0.5 mbar and 10 mbar.

In select embodiments of the present disclosure, the artificial resin comprises between about 4% w/w and about 90% w/w CBL or CBL derivative, more particularly between about 10% and about 90%, between about 25% and about 75%, or between about 40% and about 50% w/w CBL or CBL derivative. In select embodiments of the present disclosure, the artificial resin comprises about 5%, about 15%, about 25%, about 35%, about 50%, about 60%, about 75%, about 85%, about 90%, or about 95% w/w CBL or CBL derivative.

In select embodiments of the present disclosure, the first cannabinoid in the artificial resin is CBC or CBC derivative. In an embodiment, the artificial resin comprises at least about 4% w/w CBC or CBC derivative. In select embodiments of the present disclosure, the artificial resin comprises between about 4% and about 10% w/w CBC or CBC derivative, more particularly between about 10% and about 25% w/w CBC or CBC derivative, or greater than about 25% w/w CBC or CBC derivative. In select embodiments of the present disclosure, the artificial resin comprises between about 40% and about 50%, between about 50% and about 70%, or greater than about 70% w/w CBC or CBC derivative. In select embodiments of the present disclosure, the artificial resin comprises about 5%, about 10%, about 15%, about 20%, about 25%, about 30%, about 35%, about 40%, about 45%, about 50%, or more w/w CBC or CBC derivative.

In select embodiments of the present disclosure, the CBC derivative of the artificial resin is of the form:

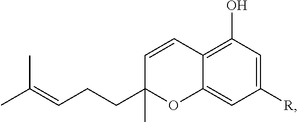

wherein R is methyl, propyl, heptyl, 1,1-dimethylheptyl, phenylethyl, or phenylvinyl.

In select embodiments of the present disclosure, the CBL derivative of the artificial resin is of the form:

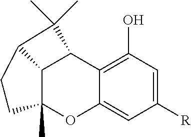

wherein R is methyl, propyl, heptyl, 1,1-dimethylheptyl, phenylethyl, or phenylvinyl. In an embodiment, the CBC derivative and CBL derivative have the same R group.

In select embodiments of the present disclosure, the second cannabinoid is a modified cannabinoid comprising the same R group as the CBC derivative and/or the CBL derivative. As used herein, the term “modified cannabinoid” refers to a natural or synthetic cannabinoid (e.g. THC, CBD, CBG, CBN, etc) having a modified substituent in place of the common pentyl chain in the position of the R group, above. A person of ordinary skill in the art, with the benefit of the present disclosure, will appreciate that CBC derivative converted to cannabinoids other than CBL derivative will also typically have the same R group as CBC derivative.

In select embodiments of the present disclosure, the second cannabinoid is any one of the cannabinoids described herein. In an embodiment, the second cannabinoid is THC (Δ9-THC), Δ8-THC, CBD, CBT, CBN, CBG, CBDV, THCA, or cannabicitran. Conversion of CBC may produce various cannabinoids. In select embodiments of the present disclosure, the second cannabinoid is cannabicitran or a cannabicitran derivative. In select embodiments of the present disclosure, the artificial resin comprises at least about 1% w/w of the second cannabinoid. In an embodiment, the artificial resin comprises between about 1% and about 25% w/w of the second cannabinoid, more particularly between about 2% and about 10% w/w of the second cannabinoid. In an embodiment, the artificial resin comprises about 1%, about 2%, about 3%, about 4%, about 5%, about 6%, about 7%, about 8%, about 9%, about 10%, or more w/w of the second cannabinoid.

In select embodiments of the present disclosure, the CBC of the starting material and/or artificial resin comprises synthetic CBC or synthetic CBC derivative. In select embodiments of the present disclosure, the starting material comprises at least 40% w/w synthetic CBC or synthetic CBC derivative, more particularly between about 40% and about 95%, between about 50% and about 95%, or between about 50% and about 75% w/w synthetic CBC or synthetic CBC derivative. In an embodiment, the starting material comprises between about 85% w/w and about 90% w/w synthetic CBC or synthetic CBC derivative. In select embodiments of the present disclosure, the CBC of the starting material comprises a CBC isolate or CBC derivative isolate.

In select embodiments of the present disclosure, the artificial resin further comprises a third cannabinoid. In select embodiments of the present disclosure, the third cannabinoid is THC or CBD. In select embodiments of the present disclosure, the artificial resin comprises at least about 1% w/w of the third cannabinoid. In an embodiment, the artificial resin comprises between about 1% and about 25% w/w of the third cannabinoid, more particularly between about 2% and about 10% w/w of the third cannabinoid. In an embodiment, the artificial resin comprises about 1%, about 2%, about 3%, about 4%, about 5%, about 6%, about 7%, about 8%, about 9%, about 10%, or more w/w of the third cannabinoid. In some embodiments, the amount of the third cannabinoid in the artificial resin is less than the amount of the second cannabinoid.

In select embodiments, the present disclosure relates to a method of producing an artificial resin, the method comprising: contacting a starting material comprising CBC or a CBC derivative with a radical initiator to form an artificial resin comprising at least about 4% w/w cannabicyclol (CBL) or CBL derivative, a first cannabinoid, and a second cannabinoid.

Non-exclusive examples of free radical initiators are described elsewhere herein. In select embodiments of the present disclosure, the radical initiator is Fe(ClO₄)₃.

In select embodiments of the present disclosure, the contacting is in the presence of a solvent. In select embodiments of the present disclosure, the solvent is a class 3 solvent. Exemplary class 3 solvents are described elsewhere herein. In select embodiments of the present disclosure, the solvent is ethyl acetate.

In select embodiments of the present disclosure, the contacting with the radical initiator is at a reaction temperature of less than about 77° C., more particularly between about 0° C. and about 77° C., between about 10° C. and about 50° C., or between about 15° C. and about 25° C. In an embodiment, the reaction temperature is about 0° C., about 5° C., about 15° C., about 20° C., about 25° C., about 30° C., about 35° C., about 40° C., about 45° C., or about 50° C.

In select embodiments of the present disclosure, the contacting with the radical initiator is for a reaction time of at least about 1 minute, more particularly between about 1 minute and about 60 minutes, between about 5 minutes and about 60 minutes, between about 10 minutes and about 30 minutes, or between about 10 minutes and about 20 minutes. In an embodiment, the reaction time is about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or longer.

In select embodiments of the present disclosure, the contacting with the radical initiator is under a reaction pressure below about 1 bar (below about 100 kPa). In an embodiment, the reaction pressure is between about 0.1 mbar and 1000 mbar, more particularly between about 0.1 mbar and 100 mbar, or between about 0.5 mbar and 10 mbar.

In select embodiments of the present disclosure, the artificial resin comprises between about 4% w/w and about 90% w/w CBL or CBL derivative, more particularly between about 10% and about 90%, between about 25% and about 75%, or between about 40% and about 50% w/w CBL or CBL derivative. In select embodiments of the present disclosure, the artificial resin comprises about 5%, about 15%, about 25%, about 35%, about 50%, about 60%, about 75%, about 85%, about 90%, or about 95% w/w CBL or CBL derivative.

In select embodiments of the present disclosure, the first cannabinoid in the artificial resin is CBC or CBC derivative. In an embodiment, the artificial resin comprises at least about 4% w/w CBC or CBC derivative. In select embodiments of the present disclosure, the artificial resin comprises between about 4% and about 10% w/w CBC or CBC derivative, more particularly between about 10% and about 25% w/w CBC or CBC derivative, or greater than about 25% w/w CBC or CBC derivative. In select embodiments of the present disclosure, the artificial resin comprises between about 40% and about 50%, between about 50% and about 70%, or greater than about 70% w/w CBC or CBC derivative. In select embodiments of the present disclosure, the artificial resin comprises about 5%, about 10%, about 15%, about 20%, about 25%, about 30%, about 35%, about 40%, about 45%, about 50%, or more w/w CBC or CBC derivative.

In select embodiments of the present disclosure, the CBC derivative of the artificial resin is of the form:

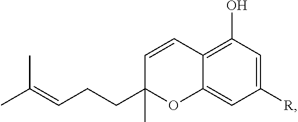

wherein R is methyl, propyl, heptyl, 1,1-dimethylheptyl, phenylethyl, or phenylvinyl.

In select embodiments of the present disclosure, the CBL derivative of the artificial resin is of the form:

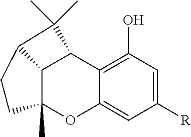

wherein R is methyl, propyl, heptyl, 1,1-dimethylheptyl, phenylethyl, or phenylvinyl. In an embodiment, the CBC derivative and CBL derivative have the same R group.

In select embodiments of the present disclosure, the second cannabinoid is a modified cannabinoid comprising the same R group as the CBC derivative and/or CBL derivative.

In select embodiments of the present disclosure, the second cannabinoid is any one of the cannabinoids described herein. In an embodiment, the second cannabinoid is THC (Δ9-THC), Δ8-THC, CBD, CBT, CBN, CBG, CBDV, THCA, or cannabicitran. In select embodiments of the present disclosure, the second cannabinoid is cannabicitran. In select embodiments of the present disclosure, the artificial resin comprises at least about 1% w/w the second cannabinoid. In an embodiment, the artificial resin comprises between about 1% and about 25% w/w of the second cannabinoid, more particularly between about 2% and about 10% w/w of the second cannabinoid. In an embodiment, the artificial resin comprises about 1%, about 2%, about 3%, about 4%, about 5%, about 6%, about 7%, about 8%, about 9%, about 10%, or more w/w of the second cannabinoid.

In select embodiments of the present disclosure, the CBC of the starting material and/or artificial resin comprises synthetic CBC or synthetic CBC derivative. In select embodiments of the present disclosure, the starting material comprises at least 40% w/w synthetic CBC or synthetic CBC derivative, more particularly between about 40% and about 95%, between about 50% and about 95%, or between about 50% and about 75% w/w synthetic CBC or synthetic CBC derivative. In an embodiment, the starting material comprises between about 85% w/w and about 90% w/w synthetic CBC or synthetic CBC derivative. In select embodiments of the present disclosure, the CBC of the starting material comprises a CBC isolate or CBC derivative isolate.

In select embodiments of the present disclosure, the artificial resin further comprises a third cannabinoid. In select embodiments of the present disclosure, the third cannabinoid is THC or CBD. In select embodiments of the present disclosure, the artificial resin comprises at least about 1% w/w of the third cannabinoid. In an embodiment, the artificial resin comprises between about 1% and about 25% w/w of the third cannabinoid, more particularly between about 2% and about 10% w/w of the third cannabinoid. In an embodiment, the artificial resin comprises about 1%, about 2%, about 3%, about 4%, about 5%, about 6%, about 7%, about 8%, about 9%, about 10%, or more w/w of the third cannabinoid. In some embodiments, the amount of the third cannabinoid in the artificial resin is less than the amount of the second cannabinoid.

In select embodiments, the present disclosure relates to a method of producing an artificial resin, the method comprising: heating a reaction mixture comprising citral, a modified resorcinol, and an amine to form a product mixture; and converting the product mixture into the artificial resin by: (i) contacting the product mixture with an acidic heterogeneous material to form CBL or CBL derivative; (ii) contacting the product mixture with a radical initiator to form CBL or CBL derivative; or (iii) irradiating the product mixture with UV light to form CBL or CBL derivative.

In select embodiments of the present disclosure, and in relation to methods for preparing an artificial resin comprising CBL, the modified resorcinol may be of the form:

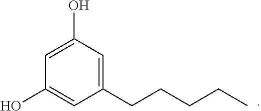

In select embodiments of the present disclosure, and in relation to methods for preparing an artificial resin comprising a CBL derivative, the modified resorcinol may be of the form:

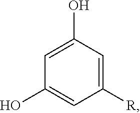

and the CBL derivative of the form:

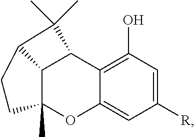

wherein R is methyl, propyl, heptyl, 1,1-dimethylheptyl, phenylethyl, or phenylvinyl. As will be appreciated by those skilled in the art who have benefitted from the teachings of the present disclosure, a derivative of CBL prepared from a modified resorcinol will typically share the same R group.

In select embodiments, the present disclosure relates to an artificial resin comprising at least about 4% CBL or CBL derivative, a first cannabinoid, and a second cannabinoid.

In select embodiments of the present disclosure, the artificial resin comprises between about 4% w/w and about 90% w/w CBL or CBL derivative, more particularly between about 10% and about 90%, between about 25% and about 75%, or between about 40% and about 50% w/w CBL or CBL derivative. In select embodiments of the present disclosure, the artificial resin comprises about 5%, about 15%, about 25%, about 35%, about 50%, about 60%, about 75%, about 85%, about 90%, or about 95% w/w CBL or CBL derivative.

In select embodiments of the present disclosure, the first cannabinoid is CBC or CBC derivative. In an embodiment, the artificial resin comprises at least about 4% w/w CBC or CBC derivative. In select embodiments of the present disclosure, the artificial resin comprises between about 4% and about 10% w/w CBC or CBC derivative, more particularly between about 10% and about 25% w/w CBC or CBC derivative, or greater than about 25% w/w CBC or CBC derivative. In select embodiments of the present disclosure, the artificial resin comprises between about 40% and about 50%, between about 50% and about 70%, or greater than about 70% w/w CBC or CBC derivative. In select embodiments of the present disclosure, the artificial resin comprises about 5%, about 10%, about 15%, about 20%, about 25%, about 30%, about 35%, about 40%, about 45%, about 50%, or more w/w CBC or CBC derivative.

In select embodiments of the present disclosure, the CBC derivative of the artificial resin is of the form:

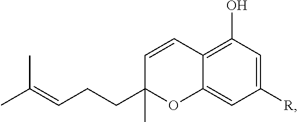

wherein R is methyl, propyl, heptyl, 1,1-dimethylheptyl, phenylethyl, or phenylvinyl.

In select embodiments of the present disclosure, the CBL derivative of the artificial resin is of the form:

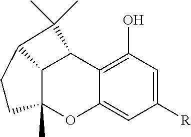

wherein R is methyl, propyl, heptyl, 1,1-dimethylheptyl, phenylethyl, or phenylvinyl. In an embodiment, the CBC derivative and CBL derivative have the same R group.

In select embodiments of the present disclosure, the second cannabinoid of the artificial resin is a modified cannabinoid comprising the same R group as the CBC derivative and/or CBL derivative.

In select embodiments of the present disclosure, the second cannabinoid is any one of the cannabinoids described herein. In an embodiment, the second cannabinoid is THC (Δ9-THC), Δ8-THC, CBD, CBT, CBN, CBG, CBDV, THCA, or cannabicitran. In select embodiments of the present disclosure, the second cannabinoid is cannabicitran or a cannabicitran derivative. In select embodiments of the present disclosure, the artificial resin comprises at least about 1% w/w of the second cannabinoid. In an embodiment, the artificial resin comprises between about 1% and about 25% w/w of the second cannabinoid, more particularly between about 2% and about 10% w/w of the second cannabinoid. In an embodiment, the artificial resin comprises about 1%, about 2%, about 3%, about 4%, about 5%, about 6%, about 7%, about 8%, about 9%, about 10%, or more w/w of the second cannabinoid.

In select embodiments of the present disclosure, the CBC of the artificial resin comprises synthetic CBC or synthetic CBC derivative. In select embodiments of the present disclosure, the CBC of the artificial resin is from a CBC isolate or CBC derivative isolate.

In select embodiments of the present disclosure, the artificial resin further comprises a third cannabinoid. In select embodiments of the present disclosure, the third cannabinoid is THC or CBD. In select embodiments of the present disclosure, the artificial resin comprises at least about 1% w/w of the third cannabinoid. In an embodiment, the artificial resin comprises between about 1% and about 25% w/w of the third cannabinoid, more particularly between about 2% and about 10% w/w of the third cannabinoid. In an embodiment, the artificial resin comprises about 1%, about 2%, about 3%, about 4%, about 5%, about 6%, about 7%, about 8%, about 9%, about 10%, or more w/w of the third cannabinoid. In some embodiments, the amount of the third cannabinoid in the artificial resin is less than the amount of the second cannabinoid.

### EXAMPLES

### Example 1

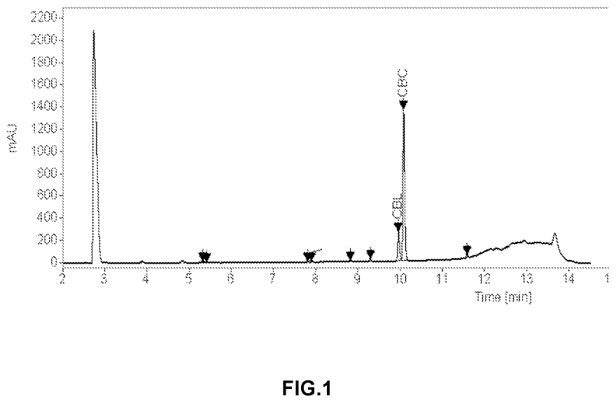

CBC (202 mg, produced synthetically from citral and olivetol) was dissolved in 48 mL 1:1 isopropyl alcohol:acetone and transferred to an 80 mL Quartz test tube. The reaction was left unstirred at room temperature for 96 hours under irradiation by a UV lamp. The solvent was evaporated to give 0.196 g of crude reaction mixture. Analysis by HPLC (FIG. 1) showed CBL as a product with a yield in the crude reaction mixture of 5% w/w.

### Example 2

CBC (250 mg, produced synthetically from citral and olivetol) was dissolved in 5 mL pentane and transferred to an 80 mL Quartz test tube. The reaction was left unstirred at room temperature for 24 hours under irradiation by a UV lamp. The solvent was evaporated to give 0.200 g of crude reaction mixture. Analysis by HPLC (not shown) showed no observable CBL as a product.

### Example 3

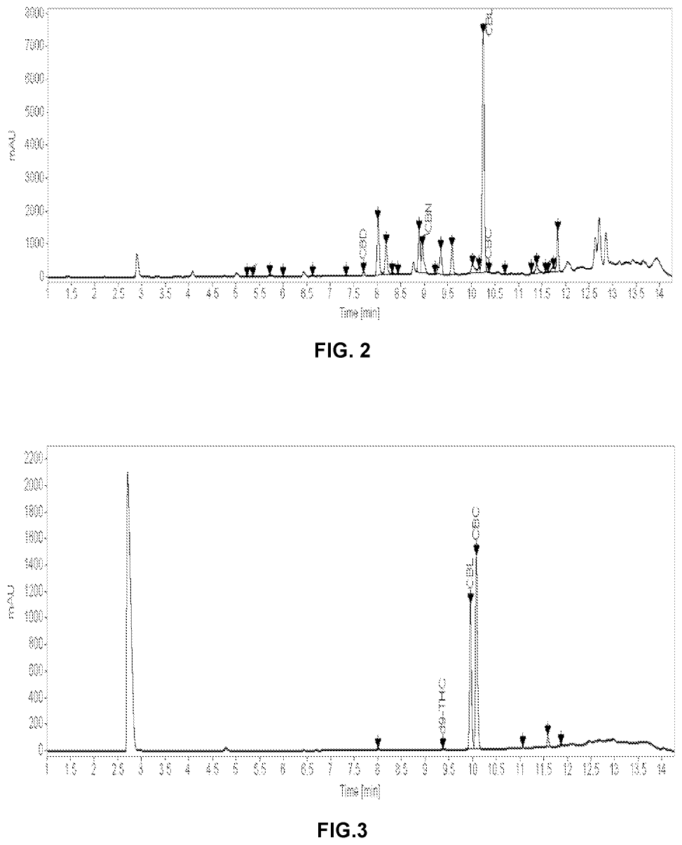

CBC (272 mg, 1 mmol) was transferred to a beaker (1 L capacity) and dissolved in 432 mL ethyl acetate followed by Fe(CI0₄)₃ (30 mg, 0.1 mmol, Sigma-Aldrich). The reaction was stirred at room temperature for 15 minutes. The solvent was evaporated to give 0.066 g of crude reaction mixture. Analysis by HPLC (FIG. 2) showed CBL as a major product with a yield in the crude reaction mixture of 16% w/w with no CBC remaining. Additional reaction time did not increase the yield of CBL.

### Example 4

CBC (250 mg, 0.8 mmol) was transferred to a round-bottomed flask (100 mL capacity) and dissolved in 13 mL chloroform followed by activated montmorillonite (250 mg, k30, Sigma-Aldrich). The montmorillonite was activated by heating in a 200° C. oven for 2 hours. The reaction was stirred for 2 hours at room temperature under nitrogen. The solvent was evaporated to give 0.196 g of crude reaction mixture. Analysis by HPLC (FIG. 3) showed CBL as a major product with a yield in the crude reaction mixture of 25% w/w with 75% w/w CBC remaining.

### Example 5

CBC (250 mg-500mg, 0.8-0.16 mmol) was transferred to a round-bottomed flask (100 mL capacity) and dissolved in 100 mL chloroform or dichloromethane followed by one of: 0.375g p-Tosyl (Sigma-Aldrich, 0.280g camphorsulfonic acid (Sigma-Aldrich), 0.256g Amberlyst-15 (Sigma-Aldrich), and 0.520g ZSM-5 (MR38, ACS). The reaction was stirred for 2 hours at 0° C. under nitrogen. The solvent was evaporated to give a crude reaction mixture. Analysis by HPLC (not shown) showed no significant CBL as a product (maximum 1% CBL w/w in product mixture).

### Example 6

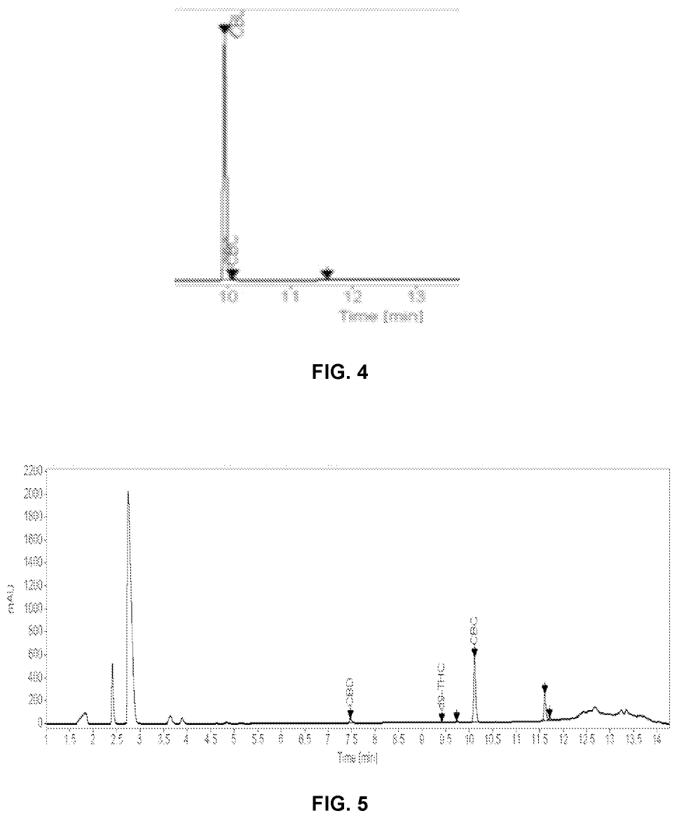

CBC (2.005 g, 6.4 mmol, 90% pure) was transferred to a round-bottomed flask (250 mL capacity) and dissolved in 100 mL chloroform followed by activated montmorillonite (2.022 g). The reaction was stirred for 48 hours at 0° C. under nitrogen. The solvent was evaporated to give 0.713 g of crude reaction mixture. The crude reaction mixture was recrystallized from heptane. Analysis by HPLC of the purified product (FIG. 4) showed CBL as a major product with a purity of 97% w/w with no CBC remaining. The yield of purified CBL was 35%.

### Example 7

To a round bottomed flask with a stopcocked side arm (100 ml capacity), was charged with 640 μL citral, 800 μL piperdine and 7.5 mL ethyl acetate. The resulting mixture was cooled to 0° C. and 800 μL acetic anhydride was added dropwise. The resulting mixture was heated at 90° C. for 1 hour, before adding a 15 mL solution toluene containing 1 g olivetol. The reaction mixture was stirred at 130° C. for 65 hours under nitrogen, after which time it was cooled to room temperature. The solvent was removed by evaporation to yield 3.1 g of crude product. Analysis by HPLC (FIG. 5) showed CBC as the major product with a yield of 30% w/w, however no CBL was observed. Replacement of 7.5 mL ethyl acetate with 15 mL toluene and 0.8 mL acetic anhydride produced no significant CBC and no observable CBL. An alternative reaction mixture of: 0.30 mL citral, 300 mg olivetol, 0.25 mL acetic anhydride, 1.8 mL toluene, and a single drop of formic acid at room temp temperature under N2 gas for 24 hours produced no significant CBC and no observable CBL.

### Example 8

A single crystal of CBL was sample was mounted on a Mitegen polyimide micromount with a small amount of Paratone N oil. All X-ray measurements were made on a Bruker Kappa Axis Apex2 diffractometer at a temperature of 110 K. The unit cell dimensions were determined from a symmetry constrained fit of 9884 reflections with 6.0°<2θ<71.12°. The data collection strategy was a number of w and cp scans which collected data up to 71.346° (2θ). The frame integration was performed using SAINT. The resulting raw data was scaled and absorption corrected using a multi-scan averaging of symmetry equivalent data using SADABS.

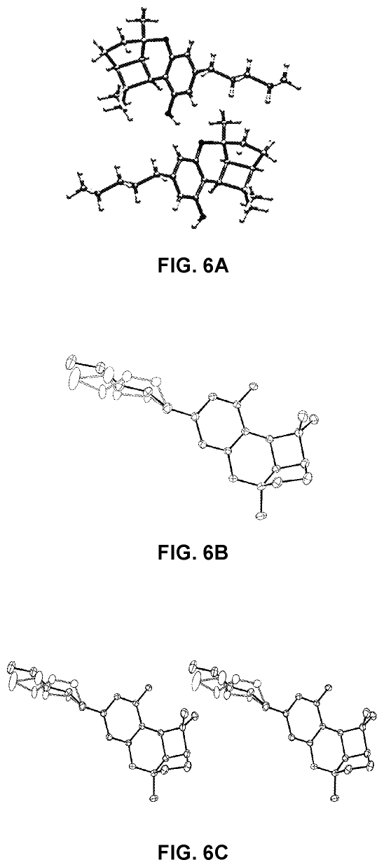

The structure was solved by using a dual space methodology using the SHELXT program. Most non-hydrogen atoms were obtained from the initial solution. The remaining atomic positions were obtained from subsequent difference Fourier maps. The hydrogen atoms were introduced at idealized positions and were allowed to ride on the parent atom. The n-pentyl group exhibited a disorder where the chain adopted three different conformations. The normalized occupancies refined to values 0.488(2), 0.345(2), and 0.167(2). The structural model was fit to the data using full matrix least-squares based on F2. The calculated structure factors included corrections for anomalous dispersion from the usual tabulation. The structure was refined using the SHELXL program from the SHELX suite of crystallographic software. A graphic plot of the structure of CBL was produced using the NRCVAX program suite and is shown in FIG. 6A. Drawings of the structure elucidated for CBL were also produced using the Oak Ridge Thermal Ellipsoid Plot (ORTEP) program. FIG. 6B shows a perspective view ORTEP drawing of a crystal structure for CBL. FIG. 6C shows a stereoscopic view ORTEP drawing of a crystal structure for CBL.

### Example 9

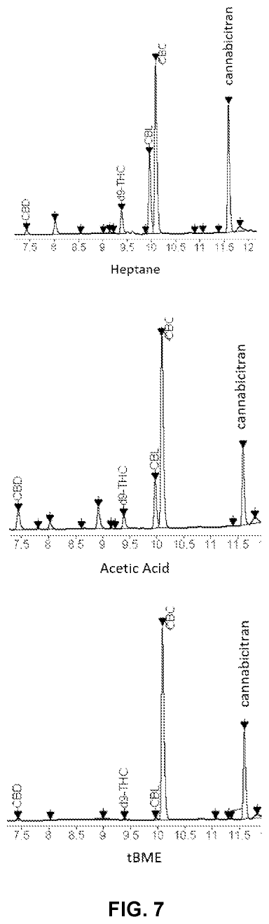

A series of reactions between CBC and Montmorillonite (K30) were performed. Unless noted otherwise below, 50 mg CBC was transferred to a round-bottomed flask (100 mL capacity) and dissolved in 2.5 mL solvent followed by activated montmorillonite (100% mass equivalent, k30, Sigma-Aldrich). The montmorillonite was activated by heating in a 200° C. oven for 2 hours. The reaction was stirred under nitrogen at a temperature and time listed in Table 1. The solvent was evaporated to give a crude reaction mixture that was analyzed by HPLC to determine the CBC, CBL, and cannabicitran content (FIG. 7 shows HPLC results for experiments 12, 15, and 16 having solvents of heptane, tert-butyl methyl ether, and acetic acid, respectively) The product compositions are shown in Table 1. Cannabicitran contents marked with “*” indicate a reaction where the CBC included 14% w/w cannabicitran. cannabicitran contents marked with a “?” contained cannabicitran at a concentration below the detection limit of the HPLC system.

TABLE 1
Reaction conditions and crude yield for Example 9.

| | | | | | Product % w/w |
|---|---|---|---|---|---|
| | Time | Temper- | | | (CBC/CBL/ |
| # | (h) | ature | Solvent | variant | cannabicitran) |
| 1 | 2 | R.T. | chloroform | 300 mg CBC, 13 mL | 73/25/2 |
| | | | | solvent | |
| 2 | 24 | R.T. | chloroform | product separation via | 4/95/trace |
| | | | | cooling to form | |
| | | | | precipitate and | |
| | | | | mother liquor | |
| 3 | | 0° C. | chloroform | | 35/25/5 |
| 4 | 24 | 0° C. | chloroform | none | 71/10/ND |
| 5 | 24 | 0° C. | chloroform | catalyst over-activated | 69/9.5/ND |
| 6 | 24 | 0° C. | chloroform | catalyst un-activated | 82/5/ND |
| 7 | 18 | R.T. | heptane | 200% Mass Eq. catalyst | 1/22/28 |
| 8 | 18 | R.T. | heptane | 50% Mass Eq. catalyst | 1/22/28 |
| 9 | 18 | R.T. | heptane | none | 67/14/4 |
| 10 | 48 | R.T. | heptane | none | 59/9/4 |
| 11 | 18 | 0° C. | heptane | 50% Mass Eq. catalyst | 65/2.5/14 |
| 12 | 18 | 0° C. | heptane | none | 46/8/15 |
| 13 | 18 | 0° C. | heptane | 200% Mass Eq. catalyst | 62/5/13 |
| 14 | 18 | 40° C. | heptane | none | 48/12/10 |
| 15 | 18 | 0° C. | tBME | none | 64/0.3/13 |
| 16 | 18 | R.T. | acetic acid | none | 40/4/7 |

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. A method for converting cannabichromene (CBC) or a CBC derivative to cannabicyclol (CBL) or a CBL derivative, the method comprising contacting the CBC or CBC derivative with an acidic heterogeneous material.

2. The method of claim 1, wherein the acidic heterogeneous material is activated montmorillonite.

3. The method of claim 1, wherein the contacting is in the presence of a solvent chosen from chloroform or a class 3 solvent.

4. (canceled)

5. The method of claim 1, wherein the contacting is at a reaction temperature of between about −20° C. and about 60° C.

6. (canceled)

7. A method for converting cannabichromene (CBC) or a CBC derivative to cannabicyclol (CBL) or a CBL derivative, the method comprising contacting the CBC or CBC derivative with a radical initiator.

8. The method of claim 7, wherein the radical initiator is Fe(ClO₄)₃.

9. The method of claim 7, wherein the contacting is in the presence of a class 3 solvent.

10. The method of claim 9, wherein the class 3 solvent is ethyl acetate.

11. (canceled)

12. The method of claim 7, wherein the contacting is at a reaction temperature of between about 15° C. and about 25° C.

13.-33. (canceled)

34. The method of claim 1, wherein the CBC or CBC derivative is a component of a cannabis extract, a cannabis distillate, a cannabis isolate, or a cannabis synthesis reaction mixture.

35. The method of claim 1, wherein the CBC derivative is of the form: 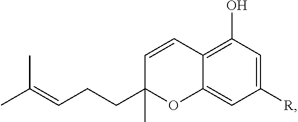
wherein R is methyl, propyl, heptyl, 1,1-dimethylheptyl, phenylethyl, or phenylvinyl.

36.-57. (canceled)

58. A method of preparing cannabicyclol (CBL) or a CBL derivative, the method comprising:
heating a reaction mixture comprising citral, a modified resorcinol, and an amine to form a first product mixture; and
(i) contacting the first product mixture with an acidic heterogeneous material to form the CBL or CBL derivative; or (ii) contacting the first product mixture with a radical initiator to form the CBL or CBL derivative.

59. The method of claim 58, which is for preparing CBL, and the modified resorcinol is of the form:

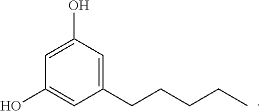

60. The method of claim 59, which is for preparing a CBL derivative of the form: 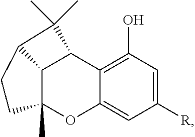
and the modified resorcinol is of the form: 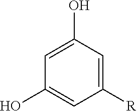
wherein R is methyl, propyl, heptyl, 1,1-dimethylheptyl, phenylethyl, or phenylvinyl.

61.-103. (canceled)

104. The method of claim 7, wherein the CBC or CBC derivative is a component of a cannabis extract, a cannabis distillate, a cannabis isolate, or a cannabis synthesis reaction mixture.

105. The method of claim 7, wherein the CBC derivative is of the form: 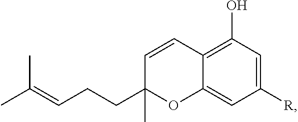
wherein R is methyl, propyl, heptyl, 1,1-dimethylheptyl, phenylethyl, or phenylvinyl.

106. The method of claim 58, wherein the acidic heterogeneous material is activated montmorillonite.

107. The method of claim 58, wherein the contacting of the first product mixture with the acidic heterogeneous material is in the presence of a solvent chosen from chloroform or a class 3 solvent.

108. The method of claim 58, wherein the radical initiator is Fe(ClO₄)₃.

109. The method of claim 58, wherein the contacting of the first product mixture with the radical initiator is in the presence of a class 3 solvent.
